(19) United States
(12) Patent Application Publication
(10) Pub. No.: US 2024/0282458 A1
(43) Pub. Date: Aug. 22, 2024

(54) METHODS OF DETERMINING AND USING A MICROBIOME RESILIENCE INDEX

(71) Applicant: SOCIETE DES PRODUITS NESTLE S.A., Vevey (CH)

(72) Inventors: FABIO MAINARDI, Lausanne (CH); MARC GARCIA-GARCERA, Ecublens (CH); ANDREA NASH, Saint-Louis, MO (US)

(21) Appl. No.: 18/687,919

(22) PCT Filed: Sep. 2, 2022
(86) PCT No.: PCT/EP2022/074427

§ 371 Date: Feb. 29, 2024

Related U.S. Application Data
(60) Provisional application No. 63/240,478, filed on Sep. 3, 2021.

(51) Int. Cl. G16H 50/30; G16H 20/60

(52) U.S. Cl. CPC G16H 50/30; G16H 20/60

(57) ABSTRACT

Methods can quantify the degree of microbiota resilience of individuals with an index, in order to be able to propose a personalized nutrition solution to those who need it, to thereby improve their microbiome resilience, especially under challenges or stressors. Preferably, a resilience index is based on analysis of the microbiota before, during, and after a challenge with high fat diet, to provide a way of quantifying the resilience status of an individual's gut microbiome, on the basis of analysis of the microbiome's composition, metabolites and other physiological parameters, during and after the challenge. The microbiome resilience index can be used for clustering of consumers as resilient or non-resilient, so that dietary intervention can be proposed and/or administered to the non-resilient individuals. The present disclosure also provides a microbiome resistance index, a microbiome recovery index, and/or a microbiome resilience index; also provides a method to screen a patient that has a low resilience; and further provides a method to screen an individual with a low resilience index and provide a recommendation such as a nutritional intervention to the individual.

### BACKGROUND

The human gut microbiome is an ecosystem of trillions of bacteria. Throughout life, the gut microbiome is challenged by one or more of unhealthy diet, antibiotics, other medications, infections, intense exercise, or alcohol. The ability of the microbiome to resist those challenges or quickly and fully recover from the perturbation is “microbiome resilience” and likely contributes to maintaining health. Reduced microbiome resilience may lead to dysbiosis with negative impact on health.

### SUMMARY

To the best knowledge of the inventors, no publication prior to the time of the present application describes a measure of assessing microbiota resilience of individual subjects.

The methods disclosed herein can quantify the degree of microbiota resilience of individuals with an index, in order to be able to propose a personalized nutrition solution to those who need it, to thereby improve their microbiome resilience, especially under challenges or stressors. In a preferred embodiment, a resilience index is based on analysis of the microbiota before, during, and after a challenge with high fat diet. This and other embodiments provide a way of quantifying the resilience status of an individual's gut microbiome, on the basis of analysis of the microbiome's composition, metabolites and other physiological parameters, during and after a challenge. The microbiome resilience index can be used for clustering of consumers as resilient or non-resilient, so that dietary intervention can be proposed and/or administered to the non-resilient individuals.

In some embodiments, the present disclosure provides a microbiome resistance index, a microbiome recovery index, and/or a microbiome resilience index; also provides a method to screen a patient who has a low resilience; and further provides a method to screen an individual with a low resilience index and provide a recommendation such as a nutritional intervention to the individual, for example a recipe or a particular food product such as a supplement.

Additional features and advantages are described herein and will be apparent from the following Figures and Detailed Description.

### BRIEF DESCRIPTION OF DRAWINGS

FIG. 1 is a schematic diagram generally illustrating microbiota resilience as defined herein.

FIG. 2A is a table showing the formulation of a non-limiting example of a suitable fiber product which can be identified and/or administered in some embodiments of the methods disclosed herein.

FIG. 2B is a table showing the formulation of a non-limiting example of a suitable probiotic product which can be identified and/or administered in some embodiments of the methods disclosed herein.

FIGS. 3, 4, 5 and 6 are graphs showing results for simulated data from the experimental example disclosed herein.

FIG. 7 is a schematic diagram showing the clinical trial design in the experimental example disclosed herein.

FIGS. 8A-8F are graphs showing example trajectories and how these trajectories are affected by changes to fPCA scores 1 and 2 according to an embodiment of the bivariate model for resilience index disclosed herein.

### DETAILED DESCRIPTION

### Definitions

Some definitions are provided hereafter. Nevertheless, definitions may be located in the “Embodiments” section below, and the above header “Definitions” does not mean that such disclosures in the “Embodiments” section are not definitions.

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number. All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this disclosure and the appended claims, the singular forms “a,” “an” and “the” include plural referents unless the context clearly dictates otherwise. Thus, for example, references to “a fiber” or “the fiber” encompass both an embodiment having a single fiber and an embodiment having two or more fibers.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified.

The terms “at least one of” and “and/or” used in the respective context of “at least one of X or Y” and “X and/or Y” should be interpreted as “X,” or “Y,” or “X and Y.” For example, “at least one of resistance or recovery” and “resistance and/or recovery” should be interpreted as “resistance,” or “recovery,” or “both resistance and recovery.”

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

“Prevention” includes reduction of risk, incidence and/or severity of a condition or disorder. The terms “treatment” and “treat” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The terms “treatment” and “treat” do not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment” and “treat” are also intended to include the potentiation or otherwise enhancement of one or more primary prophylactic or therapeutic measures. As non-limiting examples, a treatment can be performed by a patient, a caregiver, a doctor, a nurse, or another healthcare professional.

As used herein, a prophylactically or therapeutically “effective amount” is an amount that prevents a deficiency, treats a disease or medical condition in an individual, or, more generally, reduces symptoms, manages progression of the disease, or provides a nutritional, physiological, or medical benefit to the individual. The relative terms “promote,” “improve,” “increase,” “enhance” and like terms refer to resilience of the microbiome of the subject (i.e., resistance to challenge and/or recovery from challenge), after administration of the composition disclosed herein (which comprises a fiber and a probiotic), relative to the resilience of the microbiome of the subject obtained by administration of a composition lacking the fiber and/or the probiotic but otherwise identically formulated.

As used herein, the terms “food,” “food product” and “food composition” mean a product or composition that is intended for oral ingestion by a human or other mammal and comprises at least one nutrient for the human or other mammal.

“Nutritional compositions” and “nutritional products,” as used herein, include any number of food ingredients and possibly optional additional ingredients based on a functional need in the product and in full compliance with all applicable regulations. The optional ingredients may include, but are not limited to, conventional food additives, for example one or more, acidulants, additional thickeners, buffers or agents for pH adjustment, chelating agents, colorants, emulsifies, excipient, flavor agent, mineral, osmotic agents, a pharmaceutically acceptable carrier, preservatives, stabilizers, sugar, sweeteners, texturizers, and/or vitamins. The optional ingredients can be added in any suitable amount.

“Probiotic” means microbial cell preparations or components of microbial cells with a beneficial effect on the health or well-being of the host. (Salminen S, Ouwehand A. Benno Y. et al “Probiotics: how should they be defined” Trends Food Sci. Technol. 1999:10 107-10).

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

As used herein, including the appended claims, a “kit” means that the identified components are physically associated in or with one or more containers and considered a unit for manufacture, distribution, sale, or use. Containers include, but are not limited to, bags, boxes, cartons, bottles, packages of any type or design or material, over-wrap, shrink-wrap, affixed components (e.g., stapled, adhered, or the like), or combinations thereof. A single package may be one or more containers that contain the identified components, and the one or more containers are physically associated such that they are considered a unit for manufacture, distribution, sale or use.

A “subject” or “individual” is a mammal, preferably a human or companion animal. The term “companion animal” means a dog or a cat.

The “gut microbiota” is the composition of microorganisms (including bacteria, archaea and fungi) that live in the digestive tract.

The term “gut microbiome” may encompass both the “gut microbiota” and their “theatre of activity”, which may include their structural elements (nucleic acids, proteins, lipids, polysaccharides), metabolites (signalling molecules, toxins, organic, and inorganic molecules), and molecules produced by coexisting hosts and structured by the surrounding environmental conditions (see e.g. Berg, G., et al., 2020. Microbiome, 8(1), pp. 1-22).

In the present disclosures, the term “gut microbiome” may therefore be used interchangeably with the term “gut microbiota.”

### Embodiments

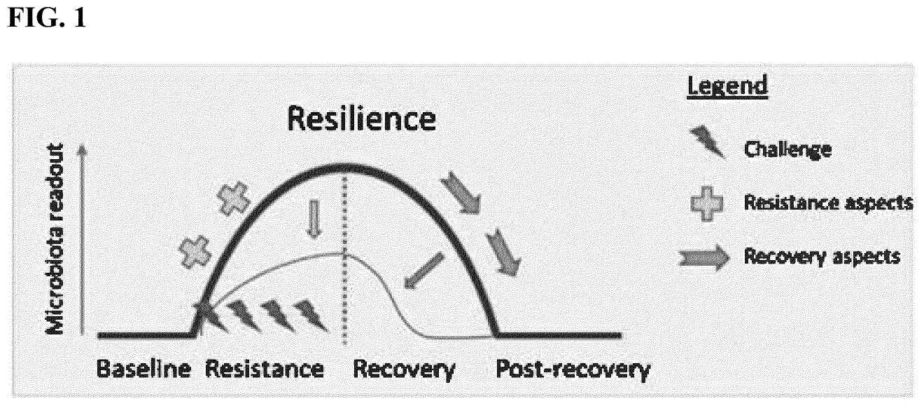

As shown in FIG. 1, microbiota resilience is defined as the capacity to resist or fully and quickly recover from challenge or perturbation. The present disclosure provides a microbiome resilience index based on analysis of the microbiota before, during, and after a challenge with high fat diet. Thus, microbiome resilience index provides a way of quantifying the resilience status of an individual's gut microbiome on the basis of analysis of its composition, metabolites and other physiological parameters, during and after a challenge.

Accordingly, in some embodiments, the present disclosure provides a microbiome resistance index, a microbiome recovery index, and/or a microbiome resilience index, and a method for determining and/or providing one or more of a microbiome resistance index, a microbiome recovery index, and/or a microbiome resilience index; also provides a method to screen a patient that has a low resilience; and further provides a method to screen an individual with a low microbiome resilience index and provide a recommendation such as a nutritional intervention to the individual, for example a recipe or a particular food product such as a supplement.

The microbiome resistance index, the microbiome recovery index, and/or the microbiome resilience index can be determined by quantifying one or more parameters of the microbiome (e.g., one or more of composition, metabolites, or other physiological parameters) before, during, and after a microbiome challenge, for example a microbiome stressor such as a high fat diet.

As used herein, a challenge with a high fat diet is a diet comprising 60-90% fat; 10-30% protein; 0-15% carbohydrates, and 0-15 g fibers per day, for at least one day, preferably at least two days, more preferably at least one three days, even more preferably at least four days, most preferably at least five days. In a particular non-limiting embodiment, a high fat diet is about 60% fat, about 25% protein, about 15% carbohydrates, and about 10 g fibers per day.

The microbiome resistance index can be determined by the following equation:

Microbiome Resistance Index ρ=1/distₘₐₓ where the individual reaches its maximum distance distₘₐₓ at a time tₘₐₓ during the challenge.

The equation for microbiome resistance index uses a baseline, a distance, and a taxonomical level; preferably baseline at day 0, Aitchison distance, and family level, respectively. The microbiome resistance index can take values in the interval (0,∞). The higher the microbiome resistance index, the more resistant is the microbiome.

The microbiome recovery index can be determined by the following equation:

$$Recovery = \frac{1}{{m}_{R}} log ( 1 + \frac{{m}_{R}}{{s}_{R}} )$$

where
mR=average distance during the recovery period,
sR=standard deviation of the distance during the recovery period.

The recovery will be higher when the average distance is closer to zero. On the other hand, if the curve does not stabilize, the term mᵣ/sᵣ tends to be bigger, so the log term in the formula represents a penalty for a lack of stability during the recovery phase.

In some embodiments, the microbiome resilience index can be determined by the following equation:

$$Resilience = \frac{{m}_{C}}{{m}_{R} \times AUC \times log ( e + {s}_{R} )}$$

where
s_{R}: standard deviation of the distance during the recovery period
AUC the area under the curve, including both challenge and recovery
m_{C} and m_{R} the average distances during the challenge and the recovery, respectively.

A high value of s_{R} will decrease the value of the index (the curve does not stabilize); a high value of AUC also leads to a low value.

Additionally or alternatively, the microbiome resilience index can be determined by a bivariate model which feeds on the Aitchison distance (b-diversity) and uses the statistical methodology of functional Principal Components Analysis (fPCA) to approximate each trajectory with a limited number of scores, according to a pre-defined approximation error, to thereby reduce the dimensionality of the data. Preferably, in embodiments which determine the microbiome resilience index by the bivariate model, the two first scores (fPCA1, fPCA2) are used to determine the microbiome resilience index, with lower values of fPCA1 and fPCA2 showing larger resilience, and higher values of fPCA1 and fPCA2 showing smaller resilience.

For example, in some embodiments, the microbiome resilience index is calculated by fPCA1 and fPCA2 from a bivariate model which uses the equations:

$${X}_{i} ( t ) = μ ( t ) + {\sum }_{k = 1}^{∞} {ξ}_{k} {ϕ}_{k} ( t )$$

$$and$$

$${ξ}_{k} = \int ( {X}_{i} ( t ) - μ ( t ) ) {ϕ}_{k} ( t ) dt .$$

An aspect of the present disclosure is a method to enhance resilience of the microbiome in a subject (e.g., a subject in need thereof). The method comprises: determining a microbiome resilience index for the subject; and proposing a nutritional intervention based at least partially on the microbiome resilience index.

The determining of the microbiome resilience index for the subject comprises quantifying one or more parameters of the microbiome (e.g., one or more of composition, metabolites and other physiological parameters) before, during, and after a microbiome challenge, for example a microbiome stressor such as a high fat diet.

The subject can be selected from the group consisting of a human infant, a human child, a human adolescent, a human adult, an elderly human, and a companion animal.

Another aspect is a method of achieving at least one result selected from the group consisting of (i) prevention or attenuation of perturbation of microbiota; (ii) recovery after perturbation; and (iii) normalization of one or more of stool frequency, intestinal transit, constipation, gut permeability, endotoxemia, or gut barrier function, the method comprising enhancing resilience of the microbiome in a subject (e.g., a subject in need thereof). The method comprises: determining a microbiome resilience index for the subject; and proposing a nutritional intervention based at least partially on the microbiome resilience index.

Yet another aspect is a method of improving gastrointestinal health, the method comprising enhancing resilience of the microbiome in a subject (e.g., a subject in need thereof) by determining a microbiome resilience index for the subject; and proposing a nutritional intervention based at least partially on the microbiome resilience index.

A related embodiment is a method of treating, preventing, reducing an incidence of, and/or reducing a severity of condition associated with a microbiome stressor in a subject who is experiencing the microbiome stressor, has recently experienced the microbiome stressor (e.g., within the most recent month or within the most recent week), and/or will experience the microbiome stressor in the near future (e.g., within the upcoming month or within the upcoming week). The method comprises: determining a microbiome resilience index for the subject; and proposing a nutritional intervention based at least partially on the microbiome resilience index.

In some embodiments, the microbiome stressor is a dietary stressor, such as a high fat diet, for example a Western diet or a ketogenic diet, or a low carbohydrate diet. As used herein, a high fat diet is a daily caloric intake in which greater than 35% of the daily caloric intake is from dietary fat, such as at least about 40% of the daily caloric intake from dietary fat, at least about 45% of the daily caloric intake from dietary fat, at least about 50% of the daily caloric intake from dietary fat, at least about 55% of the daily caloric intake from dietary fat, or at least about 60% of the daily caloric intake from dietary fat.

A Western diet is characterized by its highly processed and refined foods; high contents of sugars, salt, and fat; protein from red meat; and low content in fibers. “Low fiber” is considered as a diet with less than 15 g of fibers per 2000 calories per day.

As used herein, a low carbohydrate diet has no greater than about 15% of the daily caloric intake from carbohydrates, such as no greater than about 10% of the daily caloric intake from carbohydrates or no greater than about 5% of the daily caloric intake from carbohydrates.

In some embodiments, the subject has been consuming a high fat diet (e.g., a Western diet, or a ketogenic diet) or a low carbohydrate diet for at least one day (e.g., at least one week or at least one month) prior to a first administration of the combination of at least one fiber and at least one probiotic, with optional subsequent administrations of the combination of at least one fiber and at least one probiotic (e.g., daily administration over a time period of at least one week or at least one month).

Additionally or alternatively, the stressor can comprise one or more of: antibiotic; other medications; infections; intense exercise; stress; alcohol; travel; parenteral feeding; enteral feeding; short bowel syndrome; gut inflammation; chemotherapy; colon cancer; diarrhea; proton pump inhibitors; gluten-free diet; diet free of fermentable oligo-, di-, mono-saccharides and polyols (FODMAPs); or combinations thereof.

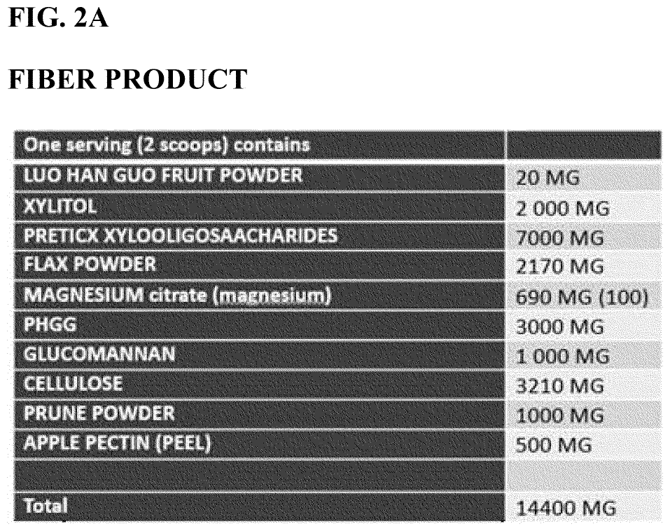

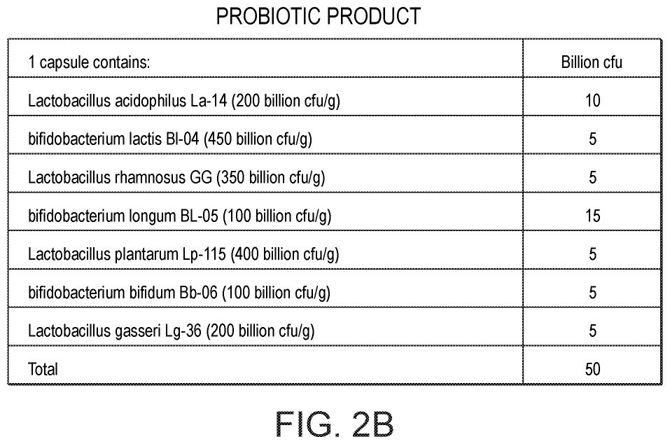

In some embodiments, the proposed nutritional intervention identifies a nutritional product, for example a composition comprising a combination of at least one fiber and at least one probiotic (e.g., in a therapeutically effective or a prophylactically effective amount). Optionally the method comprises administering the nutritional product identified by the proposed nutritional intervention. FIG. 2A shows the formulation of a non-limiting example of a suitable fiber product, and FIG. 2B shows the formulation of a suitable probiotic product.

In some embodiments, the method can comprise using the microbiome resilience index to classify the subject as resilient or non-resilient, and the dietary intervention identifies a selected composition for the non-resilient subject from one or more predetermined compositions. Preferably the selected composition is formulated to enhance microbiome resistance in the non-resilient subject.

In some embodiments, the subject consumes the nutritional product identified by the proposed nutritional intervention herein on a daily basis, for example each day for at least one week prior to a microbiome stressor or even at least one month prior to the microbiome stressor.

In preferred embodiments, each of the at least one fiber is edible, meaning that all of the components of the fibers are safe and suitable for consumption by humans and/or animals. The at least one fiber comprises insoluble fiber and/or soluble fiber, preferably a blend of insoluble fiber and soluble fiber. In some embodiments, the at least one fiber can be selected from the group consisting of xylooligosaccharides, flax seed, partially hydrolyzed guar gum (PHGG), glucomannan, cellulose, prune powder, pectin such as apple peel pectin, and mixtures thereof. In some embodiments, the at least one fiber is at least two fibers, such as two, three, four, five, six or seven fibers and optionally more. Optionally one or more of Luo Han Guo fruit powder, xylitol or magnesium (e.g., magnesium citrate) can be included with the at least one fiber.

In some embodiments, the at least one probiotic can be selected from the group consisting of Lactobacillus acidophilus, Bifidobacterium lactis, Lactobacillus rhamnosus, Bfidobacterium longum, Lactobacillus plantarum, Bfidobacterium bidum, Lactobacillus gasseri, and mixtures thereof. In some particular embodiments, the at least one probiotic can be a strain selected from the group consisting of Lactobacillus acidophilus La-14, Bfidobacterium lactis BI-04, Lactobacillus rhamnosus GG, Bfidobacterium longum BL-05, Lactobacillus plantarum Lp-115, Bifidobacterium bifidum Bb-06, Lactobacillus gasseri Lg-36, and mixtures thereof. In some embodiments, the at least one probiotic is at least two probiotic strains, such as two, three, four, five, six or seven probiotic strains and optionally more.

The at least one fiber may be administered to the individual as a total daily dose of about 5-40 g, preferably about 15-25 g. The at least one fiber may be administered in a composition comprising between about 300-1000 mg total fiber/g of dry composition.

The at least one probiotic may be administered to the individual as a daily dose of 1×10³ to 1×10¹², preferably 1×10⁷ to 1×10¹¹ cfu (cfu=colony forming unit). The at least one probiotic may be administered in a composition comprising between 1×10³ to 1×10¹² cfu/g of dry composition. The at least one probiotic may be alive, fragmented, or in the form of fermentation products (e.g., supernatant) or metabolites, or a mixture of any or all of these states.

The combination of at least one fiber and at least one probiotic is preferably orally administered in a composition such as a food composition.

The combination of at least one fiber and at least one probiotic can be administered to the individual by at least one route selected from the group consisting of oral, topical, enteral and parenteral. For example, the combination of at least one fiber and at least one probiotic can be administered in a composition selected from the group consisting of a nutritionally complete product, a drink, a dietary supplement, a meal replacement, a food additive, a supplement to a food product, a powder for dissolution, an enteral nutrition product, an infant formula, a capsule, and combinations thereof.

Optionally the combination of at least one fiber and at least one probiotic is administered in a composition further comprising at least one component selected from the group consisting of an amino acid, a protein, a nucleotide, a fish oil, a non-marine source of omega-3 fatty acids, a phytonutrient, an antioxidant, and mixtures thereof.

The composition may be a food product, an animal food product, or a pharmaceutical composition. For example, the product may be a nutritional composition, a nutraceutical, a drink, a food additive or a medicament. A food additive or a medicament may be in the form of tablets, capsules, pastilles, a liquid, or a powder in a sachet, for example.

In some embodiments, the at least one probiotic is concurrently administered in a composition separate from the at least one fiber, for example in separate compositions administered to the same individual within one hour of each other, preferably within thirty minutes of each other, more preferably within ten minutes of each other, most preferably within one minute of each other.

The composition comprising the combination of at least one fiber and at least one probiotic is preferably selected from the group consisting of milk powder based products; instant drinks; ready-to-drink formulations; nutritional powders; nutritional liquids; milk-based products, in particular yoghurts or ice cream; cereal products; beverages; water; coffee; cappuccino; malt drinks; chocolate flavoured drinks; culinary products; soups; tablets; and/or syrups.

The composition may optionally comprise any milk obtainable from animal or plant sources, such as one or more of cow's milk, human milk, sheep milk, goat milk, horse milk, camel milk, rice milk or soy milk. Additionally or alternatively, milk-derived protein fractions or colostrum may be used.

The composition comprising the combination of at least one fiber and at least one probiotic may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials.

The composition comprising the combination of at least one fiber and at least one probiotic may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. Further, the composition may contain an organic or inorganic carrier material suitable for oral or enteral administration as well as vitamins, minerals trace elements and other micronutrients in accordance with the recommendations of Government bodies such as the USRDA.

The composition comprising the combination of at least one fiber and at least one probiotic may optionally contain one or more amino acids, a protein source, a carbohydrate source and/or a lipid source, particularly in embodiments of the composition that are a food product.

Any suitable dietary protein may be used, for example animal proteins (such as milk proteins, meat proteins and egg proteins); vegetable proteins (such as soy protein, wheat protein, rice protein, and pea protein); mixtures of free amino acids; or combinations thereof. Milk proteins such as casein and whey, and soy proteins are particularly preferred.

The composition comprising the combination of at least one fiber and at least one probiotic may be administered to humans or animals, in particular companion animals, pets or livestock. It has beneficial effects for any age group. Preferably, the composition is formulated for administration to infants, juveniles, adults or elderly. In some embodiments, the composition can be administered to mothers during pregnancy and lactation to treat the infant.

The composition comprising the combination of at least one fiber and at least one probiotic can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In an embodiment, dosing is at least daily; for example, a subject may receive one or more doses daily. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

In view of the disclosures herein, an embodiment is a method to enhance resilience of the microbiome in a subject. The method comprises: determining a microbiome resilience index for the subject; and identifying a proposed nutritional intervention for the subject based at least partially on the microbiome resilience index.

Another embodiment is a method of achieving at least one result selected from the group consisting of (i) prevention or attenuation of perturbation of microbiota; (ii) recovery after perturbation of the microbiota; and (iii) normalization of one or more of stool frequency, intestinal transit, constipation, gut permeability, endotoxemia or gut barrier function. The method comprises: determining a microbiome resilience index for the subject; and identifying a proposed nutritional intervention for the subject based at least partially on the microbiome resilience index.

In some embodiments of these methods, the determining of the microbiome resilience index of the subject comprises quantifying one or more parameters of the microbiome before, during, and after a microbiome challenge. The one or more parameters of the microbiome preferably comprise one or more of composition, metabolites, or other physiological parameters of the microbiome.

In some embodiments of these methods, the microbiome challenge is a high fat diet.

In some embodiments of these methods, the proposed nutritional intervention identifies a nutritional product, preferably a nutritional product comprising at least one fiber and at least one probiotic.

In some embodiments of these methods, the subject is selected from the group consisting of a human infant, a human child, a human adolescent, a human adult, an elderly human, and an animal such as a companion animal.

In some embodiments of these methods, the method comprises classifying the subject as a resilient individual or a non-resilient individual based at least partially on the microbiome resilience index, and preferably the nutritional intervention to the non-resilient individual identifies a first nutritional product formulated to enhance resilience of the microbiome in the non-resilient individual, and optionally the nutritional intervention to the resilient individual identifies a second nutritional product which comprises at least one ingredient or amount thereof different than that of the first nutritional product.

In some embodiments of these methods, the microbiome resilience index is calculated by the equation:

$$Resilience = \frac{{m}_{C}}{{m}_{R} \times AUC \times log ( e + {s}_{R} )} .$$

Additionally or alternatively, the microbiome resilience index can be determined by a bivariate model which feeds on the Aitchison distance (b-diversity) and uses the statistical methodology of functional Principal Components Analysis (fPCA) to approximate each trajectory with a limited number of scores, according to a pre-defined approximation error, to thereby reduce the dimensionality of the data. Preferably, in embodiments which determine the microbiome resilience index by the bivariate model, the two first scores (fPCA1, fPCA2) are used to determine the microbiome resilience index, with lower values of fPCA1 and fPCA2 showing larger resilience, and higher values of fPCA1 and fPCA2 showing smaller resilience.

Further in this regard, fPCA is a specific algorithm for longitudinal data. Ina preferred embodiment of fPCA which may be used herein, Xᵢ(1) represents the value of the trajectory of individual i at time t in the following equation:

$${X}_{i} ( t ) = μ ( t ) + {\sum }_{k = 1}^{∞} {ξ}_{k} {ϕ}_{k} ( t )$$

where
μ(t) is the population average at time t
ϕₖ are the eigenfunctions of the covariance operator

The fPCA scores may be defined as:

$${ξ}_{k} = \int ( {X}_{i} ( t ) - μ ( t ) ) {ϕ}_{k} ( t ) dt$$

The above infinite sum may be truncated to the first N terms, with the higher N representing the higher the proportion of variability explained by the sum.

Accordingly, some embodiments disclosed herein use a bi-variate framework to assess the resilience of the microbiota, based on the first two fPCA scores. Notably, the scores do not take values on a fixed, standardized scale; theoretically, any negative or positive value is possible, although particularly preferred embodiments use values in the range [−10, 10].

Yet another embodiment is a method to screen a subject that has a low microbiota resilience. The method comprises determining a microbiome resilience index for the subject.

In some embodiments of the method to screen a subject, the determining of the microbiome resilience index of the subject comprises quantifying one or more parameters of the microbiome before, during, and after a microbiome challenge. The one or more parameters of the microbiome preferably comprise one or more of composition, metabolites, or other physiological parameters of the microbiome.

In some embodiments of the method to screen a subject, the microbiome challenge is a high fat diet. In some embodiments, the subject is selected from the group consisting of a human infant, a human child, a human adolescent, a human adult, an elderly human, and an animal such as a companion animal. In some embodiments, the method comprises classifying the subject as a resilient individual or a non-resilient individual based at least partially on the microbiome resilience index.

In some embodiments of the method to screen a subject, the microbiome resilience index is calculated by the equation:

$$Resilience = \frac{{m}_{C}}{{m}_{R} \times AUC \times log ( e + {s}_{R} )} .$$

Additionally or alternatively, the microbiome resilience index can be determined by one of the embodiments of the bivariate model disclosed herein, in which the two first scores (fPCA1, fPCA2) from the model may be used to determine the microbiome resilience index as follows:

$${X}_{i} ( t ) = μ ( t ) + {\sum }_{k = 1}^{∞} {ξ}_{k} {ϕ}_{k} ( t )$$

where
μ(t) is the population average at time t
ϕₖ are the eigenfunctions of the covariance operator

The fPCA scores may be defined as:

$${ξ}_{k} = \int ( {X}_{i} ( t ) - μ ( t ) ) {ϕ}_{k} ( t ) dt$$

In some embodiments of the method to screen a subject, the method further comprises comparing the microbiome resilience index for the subject to a target value or target range. For example, the interquartile range (25%-75%) of compiled data can define a “normal range,” such that a microbiome resilience index for the subject below the interquartile range of compiled data is a “low resilience,” and a microbiome resilience index for the subject above the interquartile range of compiled data is a “high resilience.” In a particular non-limiting example, the normal range of resistance is [0.6, 0.09], the normal range of recovery is [0.14, 0.20], and the normal range of resilience is [0.0023, 0.0038] (optionally rescaled to [2.3, 3.8].

Yet another embodiment is a system configured to determine a microbiome resilience index for a subject. The system comprises hardware comprising a calculation module (such as a processor) configured to calculate an abundance of selected microbiome species, use the abundance of selected microbiome species to determine the microbiome resilience index for the subject, and use the microbiome resilience index to determine a nutritional recommendation for the subject. The hardware further comprises a user interface display configured to display the nutritional recommendation for the subject and preferably also to display the microbiome resilience index for the subject. Optionally the system further comprises a DNA extraction device and/or a DNA sequencer.

In some embodiments of the system, the system is configured to receive a stool sample as input, and optionally the input further comprises one or more other parameters selected from the group consisting of age of the subject, gender of the subject, weight of the subject, a food preference of the subject, and combinations thereof. For the stool sequencing, the system preferably receives results of sequencing of at least one stool sample taken before a challenge (e.g., a high fat diet, for example for five days) and at least one sample after the challenge.

In some embodiments of the system, the calculation module is further configured to characterize the microbiome resilience index for the subject as high or low relative to a target value, and preferably the user interface display is further configured to display the characterization of the microbiome resilience index for the subject as high or low.

The system can be configured to perform the sequencing using the DNA extraction device and/or the DNA sequencer, if present, and/or can receive the result of the sequencing as input. The sequencing device optionally can be used to monitor the evolution of the microbiota resilience after the nutritional recommendation.

In some embodiments, the system is provided in a kit with one or more recipes for a high fat diet and/or one or more food products for a high fat challenge.

### Example

The following non-limiting example generally illustrates the concepts underlying the embodiments disclosed herein.

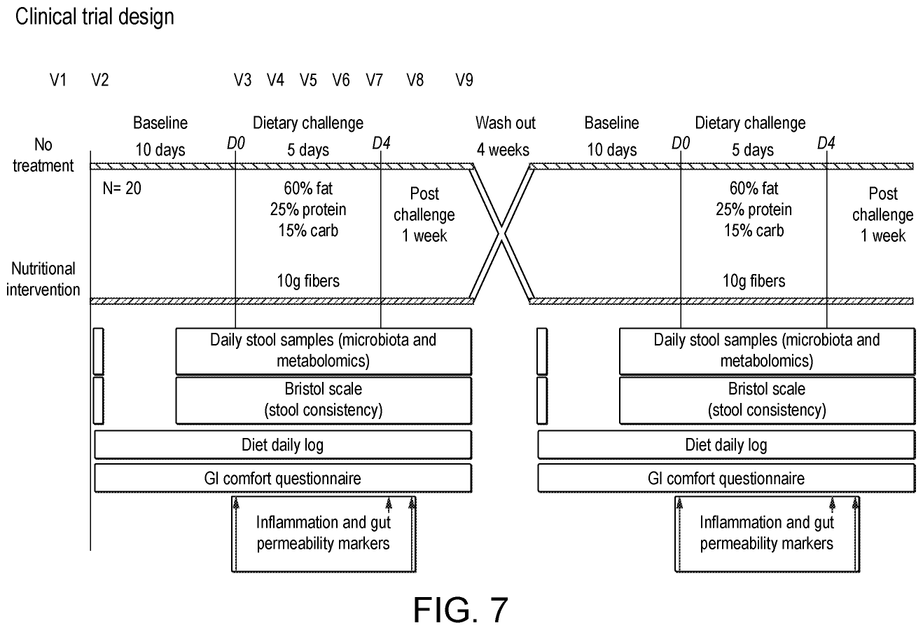

The objective was to propose formulas to quantify resistance, recovery and resilience, based on a trajectory describing the change of the microbiome over time. FIG. 7 shows the clinical trial design, which used a fiber product containing a diverse blend of fibers to improve overall microbiome diversity and a probiotic product to replenish down-regulated bacteria, diminish inflammation, and improve gut barrier. FIG. 2A shows the formulation of the fiber product, and FIG. 2B shows the formulation of the probiotic product used.

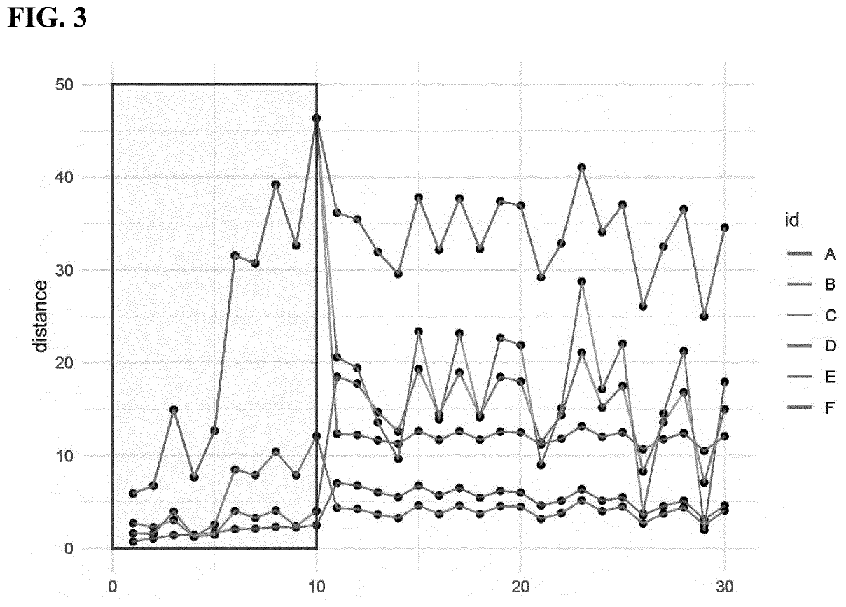

Specifically, FIG. 3 shows several curves generated, simulating different degrees of resistance, recovery and resilience.

These curves were generated as follows: days 0 to 10 simulate a challenge period, days 11 to 30 simulate a post-challenge period. For each curve, we simulated a function distanceC, to simulate the microbiota evolution during the challenge, and a function distanceR for the recovery period. These functions are detailed in the table 1 below:

TABLE 1

| Curve | distanceC | distanceR |
|---|---|---|
| A | 4*day + rnorm(N, sd = 6) | 12 + rnorm(N, sd = 1) |
| B | 4*day + rnorm(N, sd = 6) | 17 + rnorm(N, sd = 10) |
| C | 4*day + rnorm(N, sd = 6) | 34 + rnorm(N, sd = 6) |
| D | day + rnorm(N, sd = 2) | 4 + rnorm(N, sd = 1) |
| E | log(1 + day) + rnorm(N, sd = 0.1) | 10 − sqrt(day) + rnorm(N, sd = 1) |
| F | log(10 + day) + rnorm(N, sd = 1) | 20 − sqrt(day) + rnorm(N, sd = 5) |

In the table, the term rnorm(N, sd) generates N(=30) samples of a normally distributed random variable with mean 0 and standard deviation sd. This simulates a ‘noise’ component. Each function distanceC simulates an increasing distance as a function of time, with different rates of increase. However, curves A to C share the same distance function during the challenge. This allowed us to compare the recovery rate of curves sharing the same resistance. Since the formulas for recovery and resilience include a term for the variability, we simulated recovery functions distanceR with varying standard deviations.

### Summary of Results:

ITT (Intention to treat) dataset, excluding subjects with missing data at baseline:
Formulas for resistance, recovery and resilience are consistent when tested on simulated data
Resistance is higher in the intervention group, but the difference is not significant
Recovery is lower for the intervention group, but the difference is not significant
If only phase 1 is considered, recovery is lower for the intervention group (p=0.05)
Resilience is lower in the intervention group, but the difference is not significant

ITT dataset, replacing baseline with day −1 when missing:
Resistance is higher in the intervention group, but the difference is not significant (p=0.076)
Recovery is not significantly different between the two groups
Resilience is not significantly different between the two groups

### Resistance

In physics, one defines the elasticity constant of a spring by measuring its maximum displacement when subject to a force. The elasticity constant is the proportionality constant between force and displacement.

By analogy, we may think of resistance as a measure of rigidity and look at the maximum deviation from the baseline (assuming the baseline corresponds to the state of equilibrium).

Each individual reaches its maximum distance distₘₐₓ at a time tₘₐₓ during the challenge.

We define the resistance index as:

$$ρ = 1 / {dist}_{max}$$

This definition requires:
the choice of a baseline
the choice of a distance
the choice of a taxonomical level

In the examples below, the choices made were: baseline at day 0, Aitchison distance, and family level. This index can take values in the interval (0,∞). The higher the index, the more resistant is the microbiome.

### Applications to Simulated Data

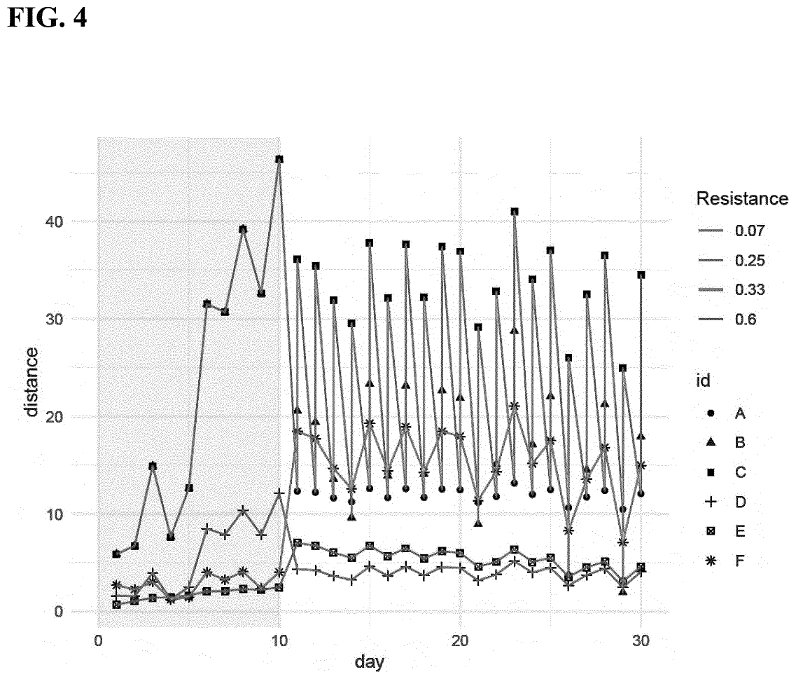

We test our definition of resistance on the simulated data introduced before (Table 2 below and FIG. 4):

TABLE 2
Resistance, for the simulated data

| id | resistance |
|---|---|
| E | 0.599 |
| F | 0.327 |
| D | 0.251 |
| A | 0.067 |
| B | 0.067 |
| C | 0.067 |

### Recovery

We define the Recovery Index as:

$$Recovery = \frac{1}{{m}_{R}} log ( 1 + \frac{{m}_{R}}{{s}_{R}} )$$

where
mR=average distance during the recovery period,
sR=standard deviation of the distance during the recovery period.

Therefore the recovery will be higher when the average distance is closer to zero. On the other hand, if the curve does not stabilize, the term mᵣ/sᵣ tends to be bigger, so the log term in the formula represents a penalty for a lack of stability during the recovery phase.

### Applications to Simulated Data

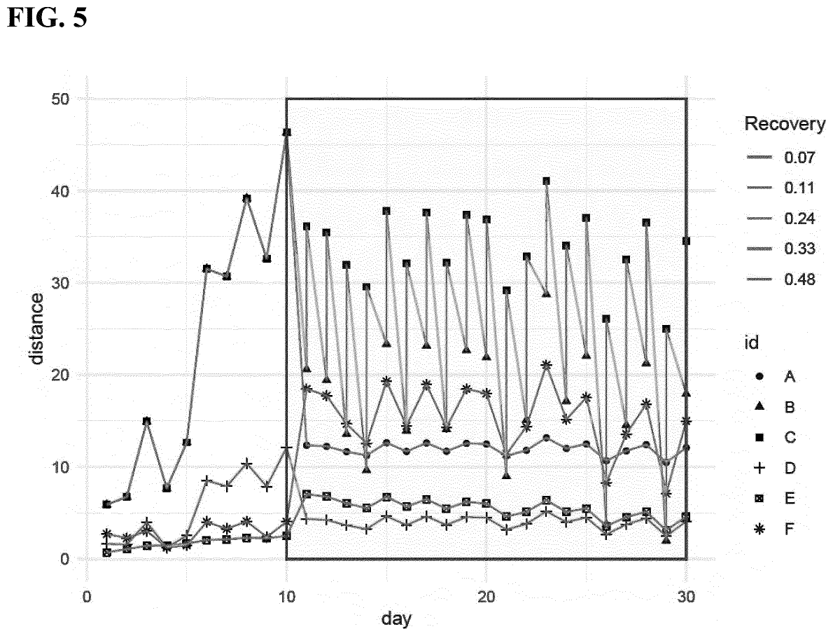

We test our definition of recovery against the simulated data introduced before (table 3 below and FIG. 5):

TABLE 3
Recovery, for the simulated data

| id | recovery |
|---|---|
| D | 0.483 |
| E | 0.334 |
| A | 0.244 |
| F | 0.108 |
| B | 0.074 |
| C | 0.066 |

### Resilience

From Dogra, S K et al. “A resilient microbiota will return to its original state of equilibrium after being subjected to a perturbation.”

Also, according to Lozupone et al, “resilience is the amount of stress or perturbation that can be tolerated before a system's trajectory changes towards a different equilibrium state.”

Note that the following definition: “resilience is the amount of stress or perturbation that can be tolerated before a system's trajectory changes towards a different equilibrium state,” is not quantifiable in practice, because it would require to measure the change in the microbiome corresponding to a series of challenges of increasing intensity.

In practice, one has a fixed challenge, and the resilience must be a property of a certain function describing the temporal variation of the microbiome, with respect to the given challenge.

We propose the following definition:
s_{R}: standard deviation of the distance during the recovery period
AUC the area under the curve, including both challenge and recovery
m_{C} and m_{R} the average distances during the challenge and the recovery, respectively

Then, we define:

$$Resilience = \frac{{m}_{C}}{{m}_{R} \times AUC \times log ( e + {s}_{R} )}$$

A high value of s_{R} will decrease the value of the index (the curve does not stabilize); a high value of AUC also leads to a low value.

### Applications to Simulated Data

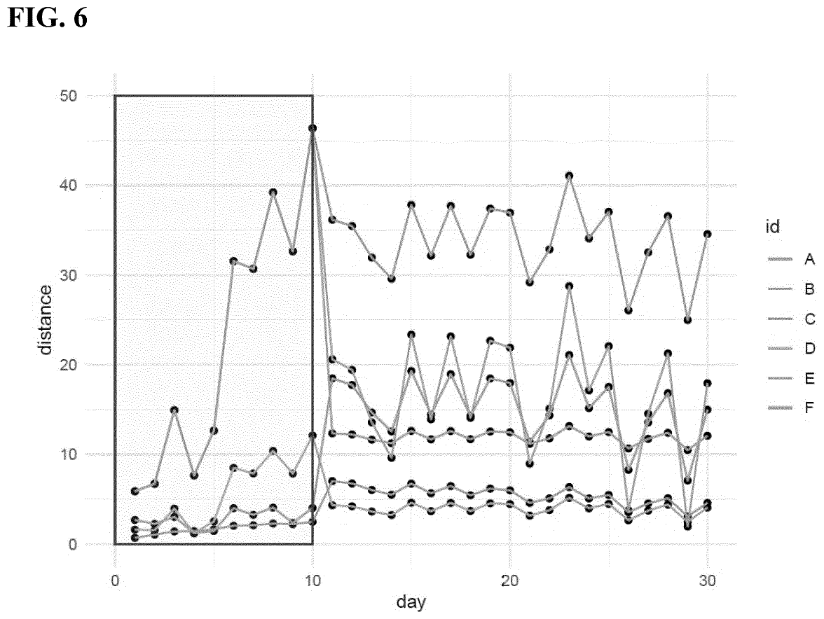

According to our index, curve is C is the less resilient, while D is the most resilient (table 4 below and FIG. 6).

TABLE 4

| id | recovery |
|---|---|
| C | 0.018 |
| F | 0.022 |
| B | 0.044 |
| E | 0.077 |
| A | 0.118 |
| D | 0.348 |

On a second iteration of the model, and of resilience, we decided to build a bivariate model to characterize the resilience of the microbiota. This model feeds on the Aitchison distance (b-diversity) and uses functional Principal Components Analysis (fPCA) to approximate each trajectory with a limited number of scores, according to a pre-defined approximation error, to thereby reduce the dimensionality of the data.

We found that the two first scores (fPCA1, fPCA2) explained 84% of the variance in the Aitchison trajectories. To evaluate the behavior of the bivariate model, we initially performed simulations of the Aitchison trajectories, based on our biological data, letting the scores fPCA1, fPCA2 vary over a grid of 2310 values, with fPCA1 varying in [−7,20] and fPCA2 in [−5, 5] (these values are the minimum and maximum observed in the data). Although both variables showed a certain degree of linkage, fPCA1 behavior reflected more the amount of disturbance that the model would withstand (resistance), while fPCA2 was impacted more by how long it would take to recover values similar to the baseline (recovery). In both cases, we observed that the lower the value of the variables, the larger the resilience of the system.

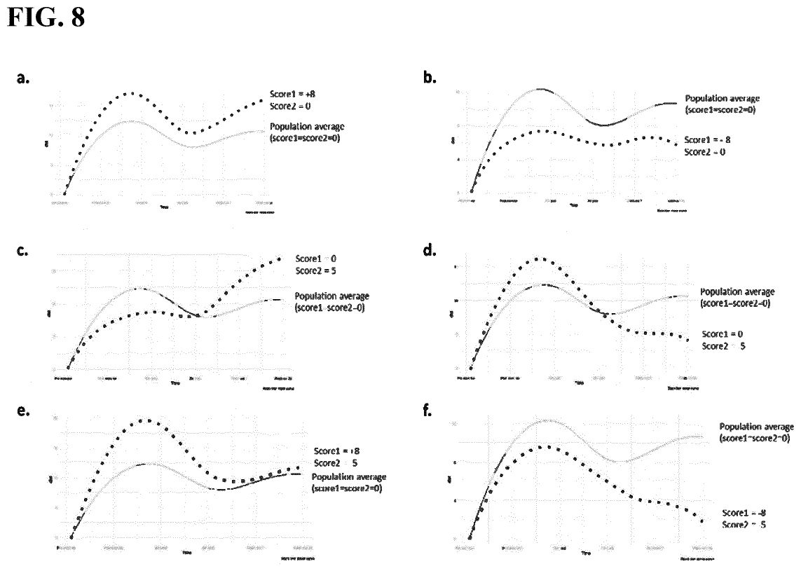

It seems that the concepts of resistance and recovery cannot be totally uncoupled; in fact, they are temporally correlated because recovery can occur after an alteration during the disturbance, so its magnitude depends how resistant is the system. FIGS. 8A-8F show example trajectories and how these trajectories are affected by changes to fPCA scores 1 and 2.

Furthermore, we compared 4 families of trajectories, arising when one of the scores is fixed at one extreme value, either minimum or maximum. So we obtained 56 curves with fPCA2=−5, obtained 56 curves with fPCA2=5, obtained 22 curves with fPCA1=−7, and obtained 22 curves with fPCA1=20. A functional ANOVA (J-T Zhang, Analysis of Variance for Functional Data, 2013) was applied to check that the mean curves of these 4 families of trajectories are statistically different from each other (p<0.01). We applied the formula 1/max(dist) to the same set of simulated trajectories, and then compared the results across the 4 families of trajectories. The pairwise comparisons were all significant.

When the models were used on the actual data, we observed a large coefficient of variation for both scores (abs(cv₁)=14.11, abs(cv₂)=9.06), consistent with the high inter-personal variation observed at compositional level. We hypothesized that the level of resilience would be impacted by the taxonomic composition, and as such, certain taxa could be more associated with higher capacity to withstand change. To test this hypothesis, we ranked the resilience scores of the participants and selected the top five (low resilience) and bottom five (high resilience) participants and compared their microbiota compositions, to extract the clades that best discriminated better both groups for each fPCA. For fPCA1, we found that members of the order Clostridiales were among the most discriminant taxa between the top and bottom participants (Wilcoxon rank test P_{fdr}<0.01). Interestingly, some of these taxa discriminating at the level of resistance (i.e., Eubacterium for high resistance) showed also a high betweenness centrality, and were differentially impacted by the nutritional challenge. On the opposite side of the spectrum, Prevotella and Butrycimonas, were associated with low levels of resistance (P_{fdr}<0.001).

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

1. A method to enhance resilience of the microbiome in a subject, the method comprising:
determining a microbiome resilience index for the subject; and
identifying a proposed nutritional intervention for the subject based at least partially on the microbiome resilience index.

2. A method of achieving at least one result selected from the group consisting of (i) prevention or attenuation of perturbation of microbiota; (ii) recovery after perturbation of the microbiota; and (iii) normalization of one or more of stool frequency, intestinal transit, constipation, gut permeability, endotoxemia or gut barrier function, the method comprising:
determining a microbiome resilience index for the subject; and
identifying a proposed nutritional intervention for the subject based at least partially on the microbiome resilience index.

3. The method of claim 1, wherein the determining of the microbiome resilience index of the subject comprises quantifying one or more parameters of the microbiome before, during, and after a microbiome challenge.

4. The method of claim 3, wherein the one or more parameters of the microbiome comprise one or more of composition, metabolites, or other physiological parameters of the microbiome.

5. The method of claim 3, wherein the microbiome challenge is a high fat diet.

6. The method of claim 1, wherein the proposed nutritional intervention identifies a nutritional product.

7. The method of claim 1, wherein the subject is selected from the group consisting of a human infant, a human child, a human adolescent, a human adult, an elderly human, and an animal.

8. The method of claim 1, comprising classifying the subject as a resilient individual or a non-resilient individual based at least partially on the microbiome resilience index.

9. The method of claim 1, wherein the microbiome resilience index is calculated by the equation:
$$Resilience = \frac{{m}_{C}}{{m}_{R} \times AUC \times log ( e + {s}_{R} )} .$$

10. The method of claim 1, wherein the microbiome resilience index is calculated by fPCA1 and fPCA2 from a bivariate model which uses the equations:
$${X}_{i} ( t ) = μ ( t ) + {\sum }_{k = 1}^{∞} {ξ}_{k} {ϕ}_{k} ( t )$$
$$and$$
$${ξ}_{k} = \int ( {X}_{i} ( t ) - μ ( t ) ) {ϕ}_{k} ( t ) dt .$$

11. A method to screen a subject that has a low microbiota resilience, the method comprising determining a microbiome resilience index for the subject.

12. The method of claim 11, wherein the determining of the microbiome resilience index of the subject comprises quantifying one or more parameters of the microbiome before, during, and after a microbiome challenge.

13. The method of claim 12, wherein the one or more parameters of the microbiome comprise one or more of composition, metabolites, or other physiological parameters of the microbiome.

14. The method of claim 12, wherein the microbiome challenge is a high fat diet.

15. The method of claim 11, wherein the subject is selected from the group consisting of a human infant, a human child, a human adolescent, a human adult, an elderly human, and an animal.

16. The method of claim 11, comprising classifying the subject as a resilient individual or a non-resilient individual based at least partially on the microbiome resilience index.

17. The method of claim 11, wherein the microbiome resilience index is calculated by the equation:
$$Resilience = \frac{{m}_{C}}{{m}_{R} \times AUC \times log ( e + {s}_{R} )} .$$

18. The method of claim 11, wherein the microbiome resilience index is calculated by fPCA1 and fPCA2 from a bivariate model which uses the equations:
$${X}_{i} ( t ) = μ ( t ) + {\sum }_{k = 1}^{∞} {ξ}_{k} {ϕ}_{k} ( t )$$
$$and$$
$${ξ}_{k} = \int ( {X}_{i} ( t ) - μ ( t ) ) {ϕ}_{k} ( t ) dt .$$

19. The method of claim 11, further comprising comparing the microbiome resilience index for the subject to a target value or target range.

20-23. (canceled)
